(19) United States
(12) Patent Application Publication
(10) Pub. No.: US 2025/0090248 A1
(43) Pub. Date: Mar. 20, 2025

(54) SYSTEMS AND METHODS FOR PROVIDING SURGICAL NAVIGATION USING MULTI-CAMERA TRACKING SYSTEMS

(71) Applicant: INTELLIJOINT SURGICAL INC., Kitchener (CA)

(72) Inventors: Ryan Visee, Mississauga (CA); Sarah Elizabeth Hynes, Summerside (CA); Andre Novomir Hladio, Waterloo (CA)

(21) Appl. No.: 18/616,706

(22) Filed: Mar. 26, 2024

Related U.S. Application Data
(60) Provisional application No. 63/539,391, filed on Sep. 20, 2023.

(51) Int. Cl. A61B 34/00; A61B 34/10; A61B 34/20; A61B 90/00; G06T 7/292; G06T 7/30; G06T 7/73; H04N 23/60

(52) U.S. Cl. CPC A61B 34/25; G06T 7/292; G06T 7/30; G06T 7/73; H04N 23/64; A61B 2034/107; A61B 2034/2048; A61B 2034/2055; A61B 2034/2072; A61B 2090/364; A61B 2090/371; G06T 2200/24; G06T 2207/30004

(57) ABSTRACT

Systems and methods perform a navigated surgical procedure involving a patient's anatomy using two or more cameras. In an embodiment, a user interface is defined and presented to guide positioning of a second camera to a target second positional relationship with a patient, the second camera having a field of view for aligning with a second surgical location, wherein a first camera is in a first positional relationship to the patient and has a field of view aligned with a first surgical location. Each of the first camera and second camera are configured to generate image data of trackers. Image data from respective cameras is received to perform the navigated surgical procedure. Further disclosed are a system and method for registering two or more cameras for use in a navigated surgical procedure and a system and method for performing a navigated surgery at two or more locations on a patient.

### CROSS-REFERENCE

The present application claims a domestic benefit of U.S. Provisional Application No. 63/539,391 filed Sep. 20, 2023, the contest of which are incorporated herein by reference it their entirety.

### FIELD

The present application relates to surgery systems and more particularly multi-camera tracking systems and methods for surgical navigation with tracked instruments.

### BACKGROUND

Navigated surgical procedures may involve relying on the identification and location of anatomical landmarks, tracking the movement and location of surgical tools (e.g., for depth of tool insertion into the surgical site) and providing navigational information and recommendations to the operating surgeon based on the anatomical landmarks. Navigational information and recommendations may consider tool location and saved positions of various anatomical landmarks, provide cut locations, pre- and post-operative spatial locations of anatomical landmarks, etc. The tools tracked and relevant anatomy may vary depending on the surgical procedure (e.g., spinal, Total Hip Arthroplasty (THA), Total Knee Arthroplasty (TKA), cranial, etc.).

Navigated surgical procedures may require an anatomical registration which may consist of navigating with respect to a reference element by identifying actual locations on the patient's anatomy (e.g., using a tracker with a locating feature such as a tip). Surgical navigation systems may also use image registration, combining navigation with pre-operative or intra-operative medical images such as computed tomography (CT) scans or X-rays, collected through various imaging modalities such as O-Arm, C-Arm, etc. These images may be registered to the surgical navigation system by correlating images to navigational information through methods such as tracking the imaging modality, identifying anatomical reference points, etc. The addition of the pre- and/or intra-operative images may allow for more precise tool navigation internally where there are more concerns for interaction between surgical tools and non-clinically relevant anatomy.

There are several modalities to enable navigated surgical procedures, including optical (monocular or stereoscopic camera systems), inertial, electromagnetic, etc. Optical navigated surgical procedures comprise camera systems that may be fixed or non-fixed and provide one or more fields of view of the surgical site. The cameras may contain additional hardware such as inertial sensors that provide additional measurements (e.g., accelerometer) and/or light emitting diodes (LEDs) that emit infrared light (IR). The cameras may contain an image sensor to detect and deliver IR light information and form an image.

Navigated surgical procedures may contain a reference element that may be the camera system in a fixed position, or an optically trackable tool (a “tracker”). The tracker may be comprised of a collection of optically trackable targets (e.g., (reflective) markers or spheres) with each optically trackable target having predetermined geometry on the tracker relative to a defined tracker origin, forming a tracker definition. Typically, a pose of the tracker is the position and orientation of the tracker in up to 6 degrees of freedom. The tracker may be fixed to other features such as a probe tip (e.g., for identifying anatomical landmarks), a surgical instrument, etc.

A computing unit may be used to communicate with the camera system (e.g., via cable, wirelessly, etc.) and the optical information (e.g., signals representative of an image) may be sent from the camera to the computing unit. The computing unit may determine the pose of the tracker in 3D space via the optical information, various forms of image processing, and spatial transformations. Various trackers may be brought in and out of the camera system's field of view, and the computing unit may provide real-time pose updates for the trackers with respect to the reference element. The pose information may be saved to provide comparative measurements, navigational recommendations and navigational information, etc. The computing unit may provide a software workflow and a user interface with a series of recommended surgical steps.

### SUMMARY

Described herein are systems and methods for performing a navigated surgical procedure involving a patient's anatomy using two or more cameras. In an embodiment, a user interface is defined and presented to guide positioning of a second camera to a target second positional relationship with a patient, the second camera having a field of view for aligning with a second surgical location, wherein a first camera is in a first positional relationship to the patient and has a field of view aligned with a first surgical location. Each of the first camera and second camera are configured to generate image data of trackers. Image data from respective cameras is received to perform the navigated surgical procedure.

In an embodiment, there is provided a system for registering two or more cameras for use in a navigated surgical procedure comprising: a first camera providing a first field of view, and configured for being positioned in a first positional relationship with a patient; a second camera providing a second field of view, and configured for being positioned independently of the first camera in a second positional relationship with the patient; a tracker for simultaneous viewing by the first and second cameras; and a computer storage device storing instructions, which when executed by a processor of a computing device, cause the computing device to: receive synchronized images of the tracker within the overlapping field of view from the first and second cameras; measure the pose of the tracker relative to the respective camera coordinate systems to calculate a registered coordinate system relative to both cameras; and provide surgical navigation relative to the registered coordinate system with the tracker in both the overlapping and non-overlapping fields of view.

In an embodiment, there is provided a system for performing a navigated surgery at two or more locations on a patient, the system comprising: (a) a first camera with a first field of view for aligning relative to the first surgical location, and configured to generate image data of trackers; (b) a second camera with a second field of view for aligning relative to the second surgical location and configured to generate image data of trackers; and (c) a computer storage device storing instructions, which when executed by a processor of a computing device, cause the computing device to: (i) receive image data from the first and second camera; (ii) detect trackers within the respective fields of view of the first and second cameras; (iii) provide a surgical workflow for the navigated surgery via a user interface; and (iv) modify the surgical workflow responsive to the detected trackers.

Computer system, method and computer program product aspects, among others, will be apparent.

### BRIEF DESCRIPTION OF DRAWINGS

FIG. 1 is an exemplary surgical navigation system with a camera, a tracker, and a computing unit, in accordance with the prior art;

FIG. 2 is a multi-camera system with an overlapping field of view, in accordance with an embodiment;

FIG. 3 is an exemplary tracker consisting of two subsets of optically trackable markers, in accordance with an embodiment;

FIG. 4A is a dual camera system mounted to a pelvis, in accordance with an embodiment;

FIG. 4B is a tracker mounted to a pelvis, in accordance with an embodiment;

FIG. 5A is a multi-camera system with an instance where the tracker is only visible in one camera, in accordance with an embodiment;

FIG. 5B is a screen of a computing unit displaying an exemplary user interface prompting the user to move the tracker so that it is within the overlapping field of view, in accordance with an embodiment;

FIG. 6 is an exemplary set up for a surgical navigation system with two camera and two surgical sites, in accordance with an embodiment;

FIG. 7A is an exemplary user interface indicating that a tracker is present at the first surgical site, in accordance with an embodiment;

FIG. 7B is an exemplary user interface indicating that a tracker is present at the second surgical site, in accordance with an embodiment;

FIG. 8A is a tracker present at the first surgical location prompting the user interface to display a set of pre-operative images belonging to the first surgical location, in accordance with an embodiment;

FIG. 8B is a tracker present at the second surgical location prompting the user interface to display a set of pre-operative images belonging to the second surgical location, in accordance with an embodiment;

FIG. 9 is an exemplary multi-camera surgical navigation system complete with a user interface providing guidance for camera placement, in accordance with an embodiment;

FIG. 10 is an exemplary image registration system that uses a first camera to relate the actual patient's pelvis to a pre-operative medical image and a user interface to provide guidance on the positioning of the second camera.

The present concept(s) is (are) best described through certain embodiments thereof, which are described herein with reference to the accompanying drawings, wherein like reference numerals refer to like features throughout. It is to be understood that the term invention, when used herein, is intended to connote the inventive concept underlying the embodiments described below and not merely the embodiments themselves. It is to be understood further that the general inventive concept is not limited to the illustrative embodiments described below and the following descriptions should be read in such light.

### DESCRIPTION

### Coregistration of 2 or More Cameras

Described herein are systems and methods for performing a navigated surgical procedure involving a patient's anatomy using two or more cameras. The scope of the claims should not be limited by the embodiments set forth in the examples but should be given the broadest interpretation consistent with the description as a whole.

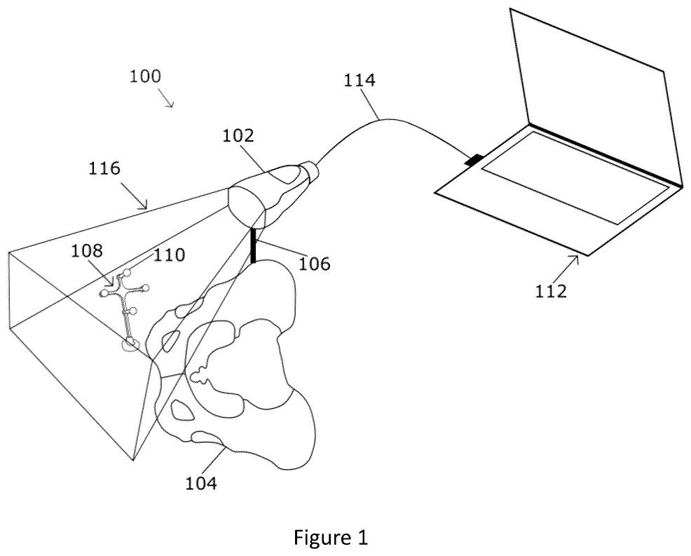

A navigated surgical procedure may consist of an optical sensor comprising a camera that acquires optical information (signals representative of an image) of a tracker that may be used to track associated instruments. Optical measurements may be provided to a computing unit and processed to provide navigational information to an operating surgeon, or to a robotic control system for controlling a surgical robot. FIG. 1 illustrates an exemplary surgical navigation system 100, in accordance with the prior art, comprising a camera 102 mounted to a pelvis 104 via a camera fixation hardware 106, and a tracker 108. The tracker may be comprised of a collection of optically trackable targets 110 (e.g., reflective markers or spheres) with each optically trackable target having a predetermined geometry (location) on the tracker relative to a defined tracker origin, forming a tracker definition.

A computing unit 112 may be a laptop, workstation, or other computing device having at least one processing unit and at least one storage device such as memory storing software. The computing unit may be configured through stored instructions. The computing unit may be coupled to the camera through a communication method (e.g., cable 114 or wirelessly) to receive optical information from the camera. The optical information may be processed by the computing unit to determine real-time pose measurements of the tracker and its optically trackable target (in up to 6 degrees of freedom), which may be used to determine navigational information. Typically, a pose of the tracker is the position and orientation of the tracker in up to 6 degrees of freedom and the tracker may be “tracked” when the pose is able to be determined within some reference frame (e.g., camera).

The camera 102 provides a field of view 116, which specifies the area in which the optically trackable target may provide a tracking signal to the camera. A tracking signal involves some form of information (e.g., reflective markers that may be processed by the camera. The tracking signal may be contained within the optical information when the optically trackable target exists within the camera field of view and is facing towards the camera. The tracker may be attached to a surgical instrument that may or may not be located within the camera's field of view but whose pose may be determined by a pre-defined positional relationship between the optically trackable target and the surgical instrument. The positional relationship may define a location of some object (e.g., optically trackable target) relative to some reference (e.g., tracker origin). A calibration technique may be used to optimize the pre-defined positional relationship between the optically trackable target and the surgical instrument and/or to fully define the positional relationship. The camera position may be defined by a coordinate system which provides a reference for the location of objects within the camera's field of view.

A rigidly fixed single camera may present challenges in the ability to track multiple surgical instruments for navigated surgical procedures over a large volume. The advantage of a two or more camera system is that a user may reduce line of sight issues and optimize coverage of the surgical area by configuring the two or more cameras in optimal positional relationships relative to one another. The scope of the mentioned “first” and “second” embodiments should not be limited to a two-embodiment system but should be interpreted to be consistent with two or more embodiment systems.

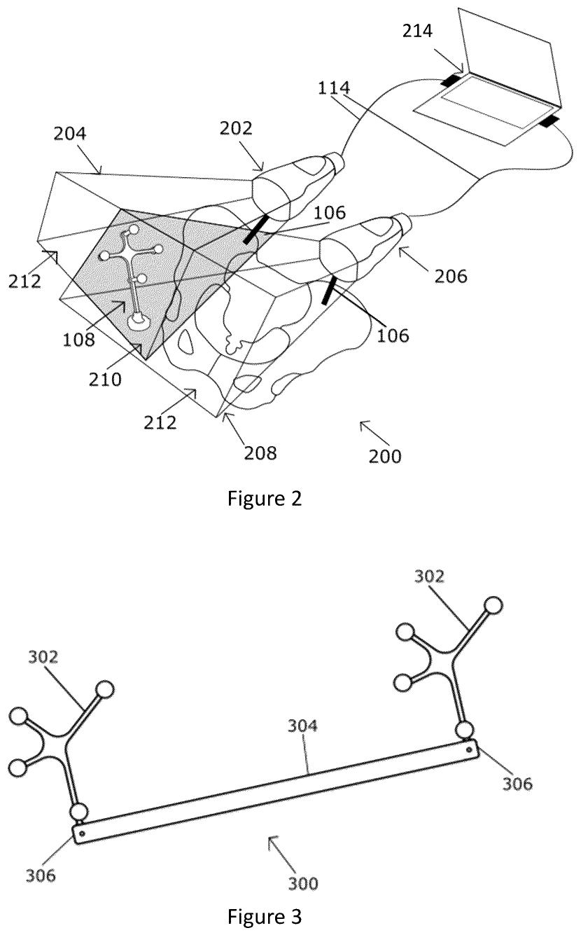

FIG. 2 provides an exemplary two or more camera system 200, in accordance with an embodiment, where a first camera 202 providing a first field of view 204 is configured to be in a first positional relationship relative to the patient, and a second camera 206 providing a second field of view 208 is configured to be in a second positional relationship relative to the camera. The first camera and the second camera may be rigidly fixed through a camera fixation hardware 106 or may be attached to the camera fixation hardware through a repeatable and accurate mount.

The first field of view of the first camera and the second field of view of the second camera may partially overlap, providing an overlapping field of view. An exemplary overlapping field of view is displayed in FIG. 2 where 210 contains the same area viewed within the respective fields of view of both the first camera and the second camera. A non-overlapping field of view 212 consists of areas within the first field of view in the first camera that may not be seen by the second camera, and vice versa.

A navigated surgical procedure that consists of the two or more camera system may comprise a computing unit 214, that is coupled to the two or more camera system through a cable 114 or by other manners (e.g. wirelessly) and receives optical information from the first camera and the second camera simultaneously. Computing unit 214 is shown as a laptop, in accordance with an embodiment, and is similar to computing unit 112 but is configured in accordance with e.g. software programming to enable operation with two or more cameras, including applicable workflow for a navigated surgical procedure such as further described.

The navigated surgical procedure may comprise a tracker 108, that comprises an optically trackable target, and that may provide a tracking signal to both the first camera and the second camera when within the first field of view and the second field of view. When in the overlapping field of view, the tracking signal may be contained within the optical information of both the first camera and the second camera and sent to the computing unit to be processed simultaneously. When in the non-overlapping field of view, the tracking signal may be contained within the optical information in only one of the first camera or the second camera.

The computing unit 214 may be configured to receive from the first camera 202, a first set of optical information, and from the second camera 206, a second set of optical information. The computing unit 214 may be configured, such as via software programming, to perform operations of a sensor fusion processing method to create a synchronized set of optical information that comprises the first set of optical information synchronized to the second set of optical information.

In one example, the sensor fusion processing method may create the synchronized set of optical information using the timestamp associated with the optical information within the first and second set of optical information. In a second example, the sensor fusion processing method may create the synchronized set of optical information using the pose of the tracker within the overlapping field of view from both the first camera and the second camera and interpolating those poses between the first and second sets of optical information. In a third example, the computing unit may be configured to provide instructions to the user (e.g. workflow via a graphical user interface (GUI) such as displayed on a display screen) to perform a set of tasks using the tracker and/or the camera hardware (e.g., light-emitting diodes (LEDs) or buttons on the camera) to create the synchronized set of optical information.

Coordinate systems define a reference framework to provide the location of objects relative to some origin. The first camera's position may be defined by a first coordinate system while the second camera's position may be defined by a second coordinate system, providing two reference frameworks that objects may be located within. As one example, an object may be located at a first location in the first coordinate system and that same object may be located at a second location in the second coordinate system simultaneously.

In an embodiment, The first camera position relative to the second camera position may be defined by a registered coordinate system, which provides a mathematical relationship between the first coordinate system and the second coordinate system, combining the first coordinate system and the second coordinate system into a single coordinate system. In the example provided above, the object that is described to be located at the first and second location within the respective first and second coordinate system, may now be described to be located at a single location within the registered coordinate system. The registered coordinate system may be determined when the optically trackable target on the tracker provides a tracking signal within a synchronized set of optical information in both the first camera and the second camera simultaneously.

In an embodiment, the synchronized set of optical information may contain the tracking signal and the computing unit may be configured to process the synchronized set of optical information and determine a pose (up to 6 degrees of freedom) of the tracker in both the first coordinate system and the second coordinate system, which provides the information necessary to determine the registered coordinate system as the same information is provided in both the first camera and the second camera. The registered coordinate system may be determined by collecting one or more poses of the tracker in the first coordinate system, collecting the same one or more poses in the second coordinate system, and using a computational method to determine the mathematical relationship (i.e. to determine the registered coordinate system) between the respective coordinate systems. The computational method may comprise an optimization operation such as iterative close point (ICP), where the Euclidean distance between the poses in the first coordinate system and the poses in the second coordinate system is minimized, and updating the registered coordinate system until an optimal solution is found. Another example of a computational method may be performing singular value decomposition (SVD) to find the optimal translation and rotation between the first coordinate system and the second coordinate system.

The computing unit may be configured to provide, in an embodiment, some calibration routine to optimize the tracker definition of the optically trackable target, providing more accuracy in the pose. One or more cameras (e.g. each as an optical sensor) may be used though a single cameras is described in the present embodiment. The calibration routine may consist of collecting optical information from the camera of the tracker using different positions, orientations, and distances (e.g. of the tracker relative to the camera), processing the optical information to calculate the pose of the tracker initially using the predefined tracker definition, and iteratively calculating a new tracker definition using an optimization process that minimizes the error in the pose calculated with the new tracker definition. The iterative optimization process continues until the optimal tracker definition is found.

For the registered coordinate system to be accurate, it may be advantageous for the first camera and the second camera to view the tracker from many different angles. In an embodiment, the system 200 (e.g. computing unit 214) may check to ensure that the tracker has been viewed from a sufficient number of different angles and may provide a warning or indication to the user if the criteria is not achieved. Such warning may be displayed, sounded or otherwise signaled to the user (e.g. vibration, etc.).

The system 200 may provide surgical navigation relative to the registered coordinate system for a surgical instrument (not shown in FIG. 2) with an optically trackable target 108 or attached to a tracker with an optically trackable target. A predefined relationship between the optically trackable target 108 and the surgical instrument may be stored within the computing unit 214 or may be determined using a calibration method, and that relationship may provide a pose relative to the registered coordinate system within the overlapping field of view 210 or the non-overlapping field of view 212. The surgical navigation may involve relying on the identification and location of anatomical landmarks, tracking the movement and location of surgical tools and providing navigational information and recommendations to the operating surgeon based on the anatomical landmarks.

The two or more camera system 200 may be positioned so that the first field of view 204 of the first camera 202 and the second field of view 208 of the second camera 206 partially overlap, creating the overlapping field of view 210, and may view the tracker 108 simultaneously within the overlapping field of view 210. The computing unit may be configured to determine the pose of the tracker 108 relative to the first coordinate system, the second coordinate system, or the registered coordinate system. As the tracker 108 is viewed simultaneously within the overlapping field of view 210, the pose of the tracker 108 may be determined simultaneously in both the first coordinate system and the second coordinate system. This information may be used to provide a more accurate pose, as the pose may be optimized to minimize the error relative to the first coordinate system and the second coordinate system, and/or may be used to update the registered coordinate system.

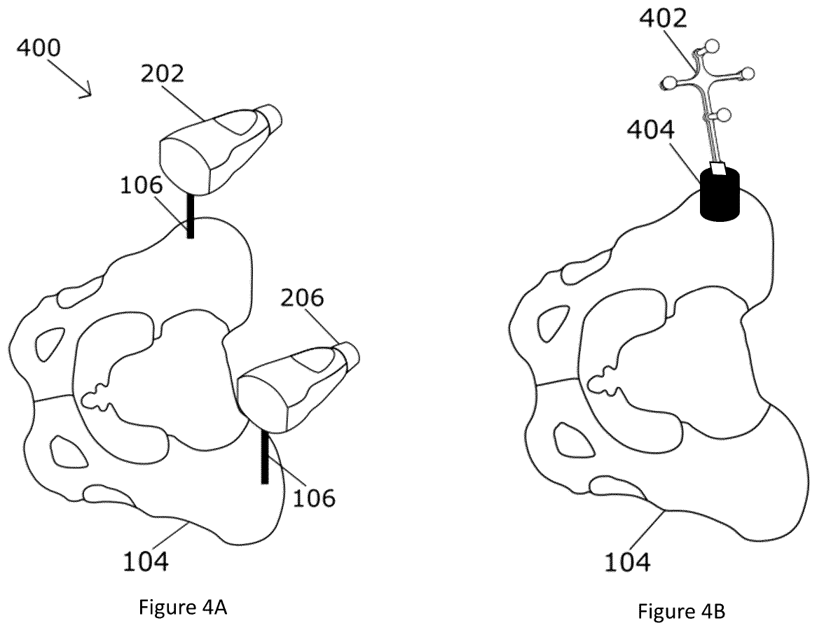

A tracker may consist of two or more subsets of optically trackable targets. An exemplary tracker 300 with two or more subsets of optically trackable targets 302 is displayed in FIG. 3 in accordance with an embodiment. The subsets of optically trackable targets 302 may or may not be identical in geometry. The subsets 302 may be fixed to a rigid body 304 and may be pre-attached or may be attached through a method for repeatable and accurate mounting, using mounts at known or unknown locations 306 relative to each of subsets 302. The rigid body may comprise one or more of a machined bar, magnet, surgical instrument, etc. The subsets 302 may be tracked by the camera system 200 by providing optical information to the computing unit 214, which may be configured to determine the pose of each subset of optically trackable targets. Each subset (e.g. 302) of optically trackable targets may have a pre-defined relationship stored in the computing unit's storage device (e.g. memory (not shown)) or may require defining the relationship during the navigated surgical procedure through the optically trackable target's respective poses or by using a calibration process. The two or more subsets of optically trackable targets 302 may have a fixed relationship on the rigid body. The computing unit 214 may provide the pre-defined fixed rigid relationship between the two or more subsets of optically trackable targets 302 in memory/storage device. The determined fixed rigid relationship between the subsets of optically trackable targets 302 may be stored in the memory of the computing unit 214. The determined relationship may be optimized using a calibration technique to increase the accuracy of the tracker 300. As an example, a calibration technique may comprise an optimization method that minimizes the error between respective poses of the subsets of optically trackable targets 302.

The two or more camera system 200 may be configured so that each camera (202, 206) may view a subset of optically trackable targets (e.g. one of subsets 302). As each camera (202, 206) in the two or more camera system 200 may only view one of the subsets 302 of the tracker 300, the cameras (202, 206) may not have overlapping fields of view 210. The two or more camera system 200 may relay optical information about each camera's respective subset of optically trackable targets to the computing unit 214 to be processed and to establish the pose of the tracker. If only one subset 302 of the tracker 300 is viewed by each respective camera (202, 206), an established pose estimation technique may be required to generate the registered coordinate system.

The first camera 202 may comprise a first inertial sensor (not shown) and the second camera 206 may comprise a second inertial sensor comprising a device (e.g. accelerometer) that measures a direction of gravity. The first inertial sensor may provide a first direction of gravity with respect to the first camera 202, while the second inertial sensor may provide a second direction of gravity with respect to the second camera 206, and the measured first and second directions of gravity may be used to calculate the registered coordinate system. The first direction of gravity and the second direction of gravity may be used to solve up to 2 degrees of freedom (both rotational) of the registered coordinate system, while the remaining 4 degrees of freedom may be solved using tracking information from the synchronized set of optical information.

In an embodiment, the computing unit 214 may be configured to receive measurements from the first inertial sensor and the second inertial sensor during a surgical navigation procedure. The computing unit 214 may calculate a relative direction of gravity between the first inertial sensor and the second inertial sensor. Differences in the first direction of gravity and the second direction of gravity to the relative direction of gravity may provide an indication of motion in the first camera 202 and/or the second camera 206. The system 200 (e.g. computing unit 214) may be configured to detect the relative motion between the first camera 202 and the second camera 206. The method to detect the relative motion may comprise of any amount of change in the measurements provided from the first inertial sensor and/or the second inertial sensor, detectable and configurable by the system.

Upon detection of relative motion between the first camera 202 and the second camera 206, the system 200 (e.g. computing unit 214) may be configured to invalidate the registered coordinate system and notify the user via a user interface that the registered coordinate system has been invalidated. Upon the invalidation of the registered coordinate system, the computing unit 214 may be configured to provide a user interface to guide the user to perform reregistration. The surgical navigation procedure may be paused until the registered coordinate system is reregistered. The user interface may also provide a warning to inform the user the registration has been invalidated and inform the user which of the first camera 202 and the second camera 206, if not both, have moved. The user interface may guide the user to position the camera that has moved into the positional relationship that satisfies the original registered coordinate system. The user interface may also inform the user to position the camera that moved into the approximate positional relationship that satisfies the original registered coordinate system and reregister the system as the tracker that was used for calculating the original registered coordinate system may still be within the camera's field of view, when positioned in the approximate original position. The user interface may inform the user of the reregistration or automatically perform reregistration without informing the user.

The system 200 (e.g. computing unit 214) may be configured to detect relative motion but may not immediately invalidate the registered coordinate system and may not immediately inform the user that the registered coordinate has been invalidated. In one example, if the first camera 202 were to be bumped by the user, causing only the first camera 202 to move relative to the second camera 206, the first camera 202 may return to the original position relative to the second camera 206 without the need for the user to interact with the system. In this case, the registered coordinate system may not need to be invalidated and the user may not need to be informed.

The computing unit 214 may be configured to monitor the motion of the first camera 202 and the second camera 206 over time by leveraging the measurements received from the first inertial sensor and the second inertial sensor, which measures the camera's direction of gravity. The computing unit 214 may be configured to receive and store the direction of gravity belonging to the first camera 202 and the second camera 206 within the storage device and determine the difference between the current direction of gravity and the previously stored direction of gravity for each the first camera 202 and the second camera 206. A difference in the first camera 202 and/or the second camera 206 direction of gravity may indicate that the first camera 202 and/or the second camera 206 has moved. The computing unit 214 may be configured to determine whether the first camera 202 and/or the second camera 206 is a fixed camera (not moving) or a non-fixed camera (moving). For example, if the first camera 202 is fixed to the patient's anatomy, the first inertial sensor may provide the computing unit 214 with directions of gravity that are not different over time, and the computing unit 214 may determine that the first camera 202 is a fixed camera. In another example, if the second camera 206 is held in the user's hand and thus is movable, the second inertial sensor may provide the computing unit 214 with directions of gravity that are different over time, and the computing unit 214 may determine that the second camera 206 is a non-fixed camera.

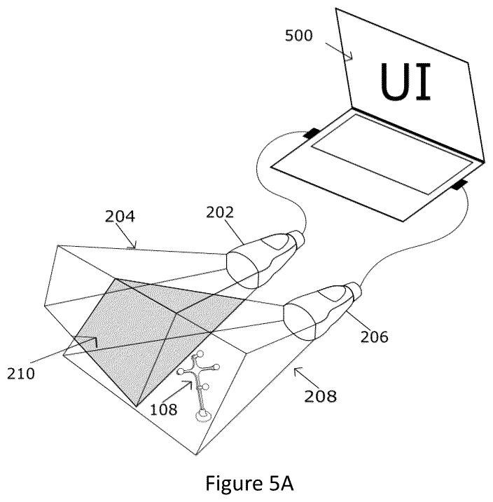

The first positional relationship and/or the second positional relationship may be a fixed relationship where the first camera 202 and/or the second camera 206 are rigidly fixed to the patient. FIG. 4A displays an exemplary setup 400 where the first camera 202 is rigidly attached to the patient's anatomy 104 through the camera fixation hardware 106 and the second camera 206 is rigidly attached to the patient anatomy through the camera fixation hardware 106. Each of first camera 202 and/or the second camera 206 may be fixed to the patient through any patient anatomy (e.g., bony structures). The camera fixation hardware 106 may comprise a fixed attachment to the camera or a repeatable attachment to the camera. The fixed relationship to the patient anatomy enables the first coordinate system to be defined relative to the patient, thus moving with the patient, in case of patient motion.

The first positional relationship and/or the second positional relationship may not be rigidly fixed to the patient when the navigated surgical procedure comprises a patient reference tracker that may be rigidly fixed to the patient and within the field of view of the first camera 202 and/or the second camera 206. FIG. 4B displays an exemplary setup, in an embodiment, where the patient reference tracker 402 is configured to be rigidly fixed to the patient anatomy 104 through the bone fixation hardware 404, providing a fixed rigid relationship between the patient reference tracker 402 and the patient anatomy 104. The fixed rigid relationship defines a coordinate system relative to the patient reference tracker 402, and thus the patient anatomy 104, instead of relative to either camera 202 or 206 (not shown in FIG. 4B). The bone fixation hardware 404 may comprise a fixed attachment to the patient reference tracker 402 or a repeatable attachment to the patient reference tracker. In an embodiment, a repeatable attachment may comprising a magnetic coupling with cooperating shaped surfaces, such as on tracker 402 and hardware 404 that urge the surfaces to form a single specific coupling (e.g. a repeatable connection). Other repeatable attachments referenced herein may be similarly configured.

The first positional relationship may be rigidly fixed to the patient anatomy 104 while the second positional relationship may be determined by the pose of the patient reference tracker 402. The registered coordinate system may be defined using the first coordinate system of the first camera 202 and the coordinate system of the patient reference tracker 402. The first positional relationship and the second positional relationship may be determined by the pose of the patient reference tracker 402. The patient reference tracker 402 may be within the overlapping field of view or the non-overlapping field of view and may consist of two or more subsets of optically trackable targets (e.g. similar to 302).

The registered coordinate system may be determined using the patient reference tracker 402. The computing unit 214 may be configured to receive the direction of gravity from the first and/or second inertial sensor and transform the received direction of gravity from the first and/or second coordinate system, into the coordinate system of the patient reference tracker 402, using the registered coordinate system. Transforming the direction of gravity may be accomplished through a mathematical model (registered coordinate system) that represents the location of objects relative to one coordinate system relative to another coordinate system. Transforming the direction of gravity into the perspective of the patient reference tracker 402 may be advantageous for the system 200 (e.g. computing unit 214) to track the orientation of the patient reference tracker 402. Movement in the patient reference tracker 402 may be defined by a difference in the orientation of the transformed direction of gravity relative to the patient reference tracker 402 and may invalidate the registered coordinate system.

The registered coordinate system may be invalid if the patient reference tracker 402 moves, and the system 200 (e.g. computing unit 214) may detect that the registered coordinate system is invalid. The system 200 (e.g. computing unit 214) may determine that the patient reference tracker 402 has moved when there is a change in the position or the orientation of the patient reference tracker 402 relative to the registered coordinate system, thus causing a difference in the registered coordinate system, and the direction of gravity of the first camera 202 and/or second camera 206 has not changed (e.g. no movement thereof). Upon detection that the patient reference tracker 402 has moved, the system 200 (e.g. computing unit 214) may provide a user interface that informs the user that the patient reference tracker 402 has moved and that the registered coordinate system has been invalidated. The user interface may inform the user how to perform reregistration or may not inform the user that reregistration is required, for example, when the reregistration is performed automatically in the background.

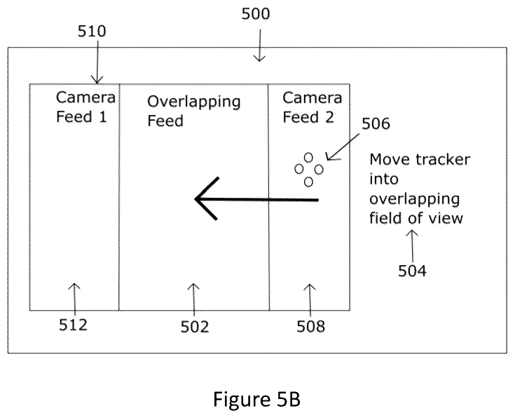

The computing unit 214 may be configured to provide a user interface that guides the user to move the tracker (e.g. 108) into the overlapping field of view 210 to be simultaneously viewed by the first camera 202 and second camera 206. The user interface comprises a signal to inform the user that the tracker 108 is not within the overlapping field of view 210, informs the user which direction to move the tracker 108 into the overlapping field of view 210, and informs the user when the tracker 108 is within the overlapping field of view. FIG. 5A illustrates a system 200 (e.g. computing unit 214) with an exemplary user interface 500 that guides the user to move the tracker 108 into the overlapping field of view 210. In FIG. 5A, the tracker is only within the second field of view 208 of the second camera 206 and not within the first field of view 204 of the first camera 202. FIG. 5B displays the exemplary user interface 500 more closely, comprising a target region 502, referencing the overlapping field of view 210, that is highlighted in one color (e.g., red) until the tracker is moved into the overlapping field of view 210, and a message 504 that informs the user to move the tracker towards the overlapping field of view 210. The tracker 108 is displayed in the user interface as the optically trackable targets 506 represented in optical information and is only contained within the non-overlapping field of view of the second camera feed 508. When the tracker 108 is moved to the overlapping field of view 210, the user may be informed through a message, popup, the target region may be highlighted a different color (e.g., green), etc.

The user interface 500 may, for example, comprise an image that displays a merged image feed 510 of both the optical information from the first camera 512+502 and the second camera 508+502, displaying both the overlapping feed 502 representing field of view 210 with the non-overlapping feeds 508 and 512 showing respective non-overlapping fields of view 212, based on the registered coordinate system. The merged image feed may overlay only the image as the overlapping feed. The overlapping feed 502 may be highlighted differently compared to the non-overlapping feed, informing the user of a target area. The displayed target area and/or the merged image feed may vary based on the amount of the overlapping field of view 210.

The merged image feed may be based on the perspective of the first camera 202 or the second camera 206, or on another perspective that is relative to the registered coordinate system (e.g. patient reference tracker 108). The registered coordinate system may be used to merge the respective image feeds through transforming the image coordinates of one image into the coordinate system of the other image reference frame (camera). Transforming coordinates may be accomplished through a mathematical model (registered coordinate system) that represents the location of objects relative to one coordinate system relative to another coordinate system. The registered coordinate system may be used to merge the respective image feeds by transforming the image coordinates of both images into the coordinate system of another reference frame (e.g., patient reference tracker 108). The merged image feed may be generated using the tracking signal contained within the optical information of the first camera 202 and the second camera 206 within the overlapping field of view 210 to merge the respective image feeds, using an image processing technique (e.g., feature extraction and matching, homography, etc.). The user interface may, for example, be configured to provide a guided model to inform the user how to put the tracker 108 into the overlapping field of view 210, relative to the provided perspective.

In an embodiment, the system 200 (e.g. computing unit 214) may be configured to perform an anatomical registration and/or an image registration using a registration tracker with optically trackable targets. Anatomical registration may consist of creating a coordinate system relative to a reference element (e.g., camera 202/206, patient reference tracker 108, etc.) by identifying a plurality of locations on the actual patient's anatomy 104, and the plurality of locations may be collected, for example, by a tracker (e.g. 108) attached to a surgical instrument (not shown). The coordinate system may then correspond to the location of the patient anatomy 104. The computing unit 214 may access calibration data to define the positional relationship between the optically trackable target (e.g. like 302) on the tracker and the surgical instrument to determine the surgical instrument's relative pose. The calibration data may be pre-defined or may be calculated through a calibration routine.

Image registration is defined through a mathematical model and that mathematical model is a coordinate mapping between a reference element and an image. Image registration may comprise generating (e.g. by computing unit 214) a coordinate system using medical images (e.g. x-rays, CT-scans, etc.), choosing a plurality of landmarks corresponding to patient anatomy on the medical images, and relating those images to the plurality of locations collected on the actual patient's anatomy. The registration tracker may collect the plurality of landmarks, which may identify actual locations on patient anatomy that correspond to the plurality of landmarks chosen on the medical image, based on image data that may be received by the computing unit. In both the anatomical registration and the image registration, the plurality of landmarks from the actual patient anatomy may be processed by the computing unit to generate the anatomical registration and/or image registration.

The computing unit 214 may be configured to receive the plurality of landmarks contained within optical information from both the first camera 202 and the second camera 206 to generate the anatomical and/or image registration when the registration tracker is within the overlapping field of view. When the registration tracker is not within the overlapping field of view, the computing unit 214 may be configured to receive a first subset of the plurality of landmarks from the first camera 202 and a second subset of the plurality of landmarks from the second camera 206, and all the plurality of landmarks may be transformed into the perspective of the registered coordinate system to generate the anatomical and/or image registration.

During the surgical navigation procedure, a tracker (e.g. 108) may transition from the first field of view 204 to the second field of view 208, or vice versa. During this transition, the pose of the tracker 108 in the second field of view 208 may be based on the pose of the tracker 108 in the first field of view 204 and transformed using the registered coordinate system. The tracker 108 may transition from the overlapping field of view 210 to the non-overlapping field of view (e.g. one of 212) and the optically trackable targets on the tracker 108 may be fully visible in the first field of view 204 and partially visible in the second field of view 208, or vice versa. As the registered coordinate system is known, the position of the optically trackable targets in the second field of view 208 may be estimated, based on the pose of the tracker 108 in the first field of view system 200 (e.g. computing unit 214), transformed using the registered coordinate system. The identified tracker 108 and identity of the optically trackable targets from the first field of view system 200 (e.g. computing unit 214) may be assigned to the partially visible optically trackable targets in the second field of view 208, improving the robustness and accuracy of tracking during transition between the first field of view 204 to the second field of view 208, and vice versa.

The tracker 108 may transition from the first field of view 204 to the second field of view 208, and vice versa, while both field of views are non-overlapping, and the optically trackable targets may be partially visible in the first field of view 204 and partially visible in the second field of view 208, and the pose may be estimated using the registered coordinate system.

The optically trackable targets on the tracker 108 may completely transition from being fully visible in the first field of view 204 to being fully visible in the second field of view 208, or vice versa, and the expected position of the tracker 108 in the second field of view 208 may be estimated, using the registered coordinate system. The identified tracker 108 and identity of the optically trackable targets from the first field of view 204 may be assigned to the visible optically trackable targets in the second field of view 208.

### Systems and Methods Using 2 or More Cameras for Performing Surgery at 2 or More Sites on a Patient's Body

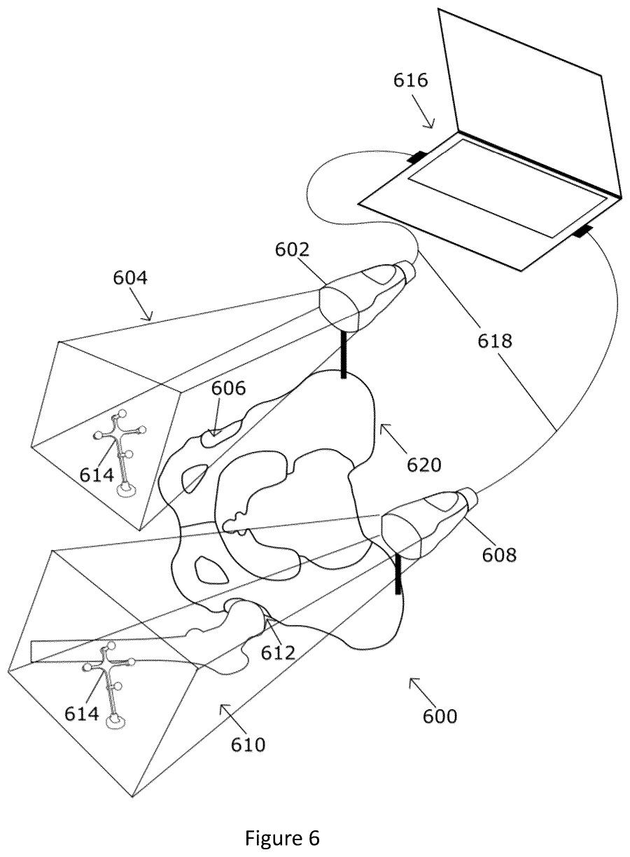

A navigated surgical procedure may comprise two or more cameras, providing two or more fields of view, at two or more locations on a patient. FIG. 6 illustrates an exemplary system 600 that provides two or more navigated surgical procedures on two or more locations on a patient, where a first camera 602 (e.g. similar to camera 202) providing a first field of view 604 for aligning relative to a first surgical location 606 and a second camera 608 (similar to camera 206) providing a second field of view 610 for aligning to a second surgical location 612. In the example provided, the first surgical location 606 may correspond to one hip joint of the pelvis 620, while the second surgical location 612 may correspond to the other hip joint on the pelvis 620 but should be interpreted to apply to most general surgical procedures and parts of the body. The first camera 602, configured to generate image data of trackers 614 and the second camera 608, configured to generate image data of trackers 614, may be connected to a computing unit 616 (similar to unit 214, with applicable software programming as described) via cables 618 (similar to cables 114) or some other manner (e.g., wirelessly), that may be configured to receive image data from the first camera 602 and the second camera 608.

The computing unit 616 may contain a computer storage device (e.g. memory (not shown)) storing instructions that when executed by one or more processors (not shown) of the computing unit 616, cause the computing unit to perform operations of one or more methods. For example, such instructions configure the computing unit 616 to receive image data from the first camera 602 and the second camera 608, and detect the trackers 614, which comprises identifying if a tracker 614 is within the first field of view 604 and/or the second field of view 610 and, if so, identify the tracker 614 and provide a pose thereof (up to 6 degrees of freedom). The trackers 614 may be identified through varying tracker definitions and/or tracker identifiers. The computing unit 616 may be configured to identify and determine poses for trackers 614 unique to the first camera 602, within the first field of view 604 and/or identify and determine poses for trackers 614 unique to the second camera 608, within the second field of view 610.

In an embodiment, the trackers 614 comprise multiple uniquely identifiable trackers (not shown), each with optically trackable targets in varying tracker definitions and/or with varying identifiers. For example, a first reference tracker (not shown) may have a first pre-defined tracker definition based on the geometry of the optically trackable objects while a second reference tracker (not shown) may have a second pre-defined tracker definition based on the geometry of the optically trackable objects that is different than the tracker definition of the first reference tracker. These trackers may undergo calibration techniques or uniqueness techniques to ensure the geometries are accurate and unique so that a robust pose may be calculated. The first reference tracker may have a first identifier (e.g. extra optically trackable target, pattern, etc.) and the second reference tracker may have a second identifier that is different from the first identifier. The trackers may be the same if the first field of view never overlaps with the second field of view, providing that the trackers seen by the first camera 602, or the second camera 608 are specific to the first surgical location or the second surgical location.

In an embodiment, computing unit 616 may be configured to recognize certain trackers from information received from the cameras 602, 608, for example, if the trackers have a specific geometry, identifier, etc. In one example, there may be two trackers, tracker A and tracker B, and the first camera 602 may only recognize tracker A and not tracker B, while the second camera 608 may only recognize tracker B and not tracker A. That is the computing unit 616 recognizes tracker A only in information from camera 602 and recognizes tracker B only in information from camera 604. Therefore, tracker B may be within the first field of view 604 of the first camera 602 but a pose for tracker B will not be determined, unless tracker B is also within the second field of view 610 of the second camera 608.

In an embodiment, the cameras 602, 608 computing unit 616 may be configured to identify and determine poses of trackers (from information from either of the cameras 602, 608) that are not unique to the first camera 602 and/or second camera 608. In one example, there may be two trackers, tracker A and tracker B, and both the first camera 602 and the second camera 608 provide information and computing unit 616 is configured to identify when either tracker A and/or tracker B are within the field of view (604 or 610) of the first 602 and/or second camera 608. Therefore, tracker A and/or tracker B may be within the field of view (604 or 610) of the first 602 and/or second camera 608 and a pose for tracker A and/or tracker B will be determined. These examples should not be limited to the scope of two trackers and two cameras but should be interpreted for two or more trackers and two or more cameras.

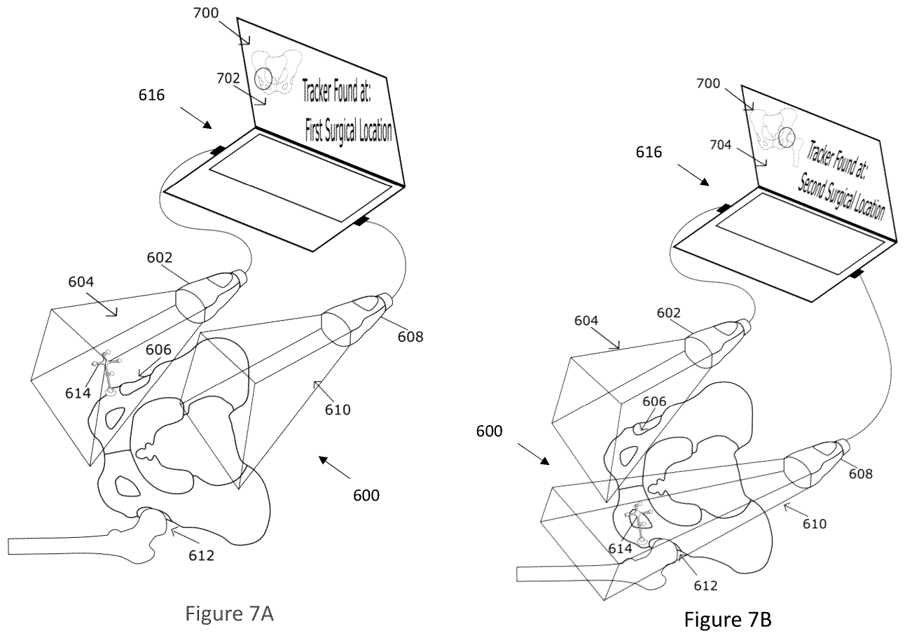

In an embodiment, a computing unit 616 may be configured to provide a surgical workflow for the first 606 and/or second surgical location 612 in the system 600 through a user interface. System 600 may be configured (e.g. via software programming, etc.) to perform operations of a method to detect trackers 614 that may be associated with the respective surgical workflow and update the user interface accordingly. FIG. 7A shows system 600 displaying an exemplary user interface 700 where the first field of view 604 contains the tracker 614 that is associated with the first surgical location 606 and the second field of view 610 does not contain any trackers therewithin, and therefore the user interface 700 displays a first surgical workflow 702 for the first surgical location 606 and does not display a second workflow for the second surgical location 612. FIG. 7B shows system 600 displaying an exemplary user interface 700 where the first field of view 604 does not contain any trackers 614 therewithin and the second field of view 610 contains the tracker 614 therewithin that is associated with the second surgical location 612. Therefore the user interface 700 displays the second surgical workflow 704 for the second surgical location 612 and does not display the workflow for the first surgical location 606. The trackers (e.g. 614) may comprise attachments to surgical instruments (not shown) associated with the surgical procedure and therefore unique trackers for cameras 602 and 608 also includes unique instruments, based on the respective tracker 614 attached to the instrument. The tracker 614 may be detectable in both the first 604 and second field of view 610, and updating the user interface may be determined when the tracker 614 is detected either the first 604 or second field of view 610. The trackers 614 may be multiple uniquely identifiable trackers that are unique to either the first 604 or second field of view 610.

In an embodiment, the system 600 may contain instances where the first field of view 604 contains a first tracker 614 that is associated with the first surgical workflow and the second field of view 610 contains a second tracker 614 that is associated with the second surgical workflow simultaneously. The system 600 (e.g. computing unit 616) may be configured to choose which surgical workflow to display, may display both surgical workflows, and/or the surgical workflow may be chosen by the user and displayed via the user interface. The system 600 (e.g. computing unit 616) may be configured to use some form heuristics to determine which workflow to display via the user interface. The system 600 (e.g. computing unit 616) may respond to a parent surgical workflow to choose which workflow to display via the user interface that, for example, may be based on the expected order of the respective surgical workflows.

The system 600 (e.g. computing unit 616) may be configured to ignore the trackers 614 that are not associated with the first 606 and/or second surgical location 612. For example, a tracker 614 may have an optically trackable target whose tracker definition is not identifiable by the first camera 602 but is identifiable by the second camera 608, or vice versa. The system 600 (e.g. computing unit 616) may be configured to inform the user that the tracker 614 identified in the first camera 602 and/or the second camera 608 does not “belong” to the first camera 602 and/or the second camera 608, through the user interface. For example, to notify that such a tracker 614 is not one that is for use with a present workflow or portion thereof. The system 600 (e.g. computing unit 616) may be configured to ignore one or more of the trackers 614 based on the geometry of the optically trackable objects thereon and/or based on the tracker identification.

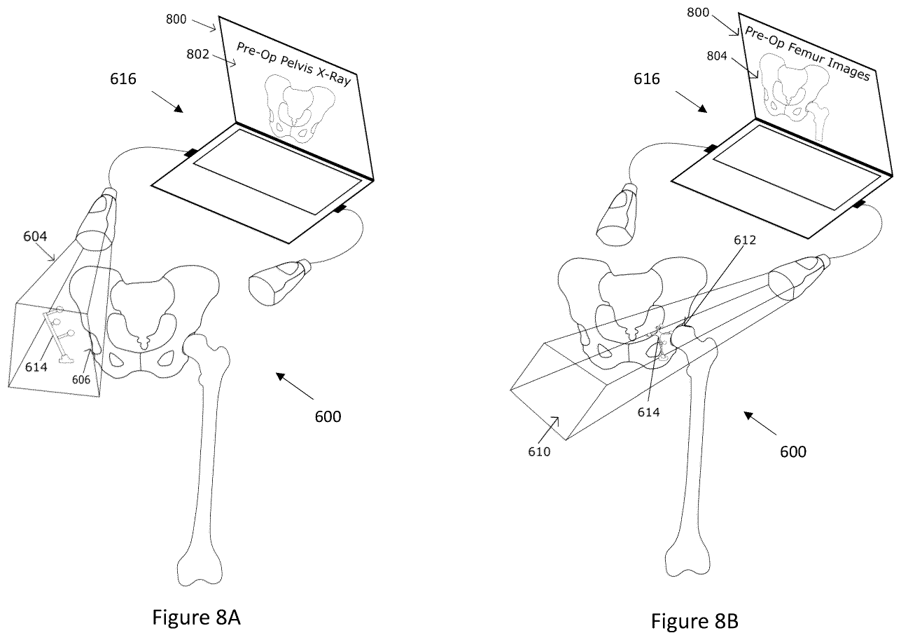

In an embodiment, the system 600 that provides two or more navigated surgeries may be the image-based navigated procedure, using medical images and relating those images to a plurality of landmarks on the patient anatomy, and the medical images on the user interface may be modified when the surgical workflow changes, based on the detected trackers 614 within the first field of view 602 and/or the second field of view 610. FIG. 8A illustrates system 600 in an embodiment, displaying an exemplary user interface 800 where the tracker 614 that may be identified in the first field of view 604 and is associated with the first surgical location 606, is identified, thus displaying the set of images 802 that belong to the first surgical location 606. FIG. 8B illustrates system 600 in an embodiment, where the tracker 614 is contained within the second field of view 610 and is associated with the second surgical location 612. Thus the user interface 800 displays the set of images 804 that belong to the second surgical location 612. The system 600 may be configured (e.g. the computing unit 616) to provide choice as toe which set of images to display or may display both sets of images, if both the first field of view 604 and the second field of view 610 contain a tracker 614 that is detectable within the respective field of view. The tracker 614 may be detectable in both the first 604 and second field of view 610, and the medical images displayed via the user interface may be determined when the tracker 614 is detected either the first or second field of view. The trackers may be multiple uniquely identifiable trackers that are unique to either the first 604 and second field of view 610.

In an embodiment, the first camera 602 may comprise a first inertial sensor (not shown) and the second camera 610 may comprise a second inertial sensor (not shown). Each inertial sensor comprises a device (e.g., accelerometer) measuring a direction of gravity. In an embodiment, the surgical workflow may be modified based on the measurements the system 600 (e.g. computing unit 614) receives from the first inertial sensor and/or the second inertial sensor. As one example, the first surgical location 606 may be located posteriorly on the patient anatomy 104 and the second surgical location 612 may be located laterally on the patient anatomy 104, and the patient anatomy 104 may be positioned supine or prone. During this surgical procedure, the first camera 602 may be placed in a first positional relationship with a base of the first camera 602 pointing toward the direction of gravity, while the second camera 608 may be placed in a second positional relationship with the side of the second camera 608 pointing toward the direction of gravity (e.g. where the second camera 608 and the first camera 602 are relatively tilted by 90°). The surgical workflow may be modified based on the inertial measurements received from either the first inertial sensor and/or the second inertial sensor, compared to the expected gravity vector for either the first surgical location and/or the second surgical location. This may be advantageous for trackers that are not detectable by both cameras 602 and 608 and may be tracked in either the first field of view 604 or second field of view 610, as the active surgical location (606 or 612) may be based solely on the inertial measurement.

In an embodiment, the system 600 may monitor the inertial measurements from the first inertial sensor and/or the second inertial sensor to detect the position of the patient anatomy 104 and use that position as an indicator of which navigated surgical procedure is being performed. The surgical workflow and/or the user interface may be modified based on the detected navigated surgical procedure. As an example, this may occur in surgical procedures where the patient anatomy 104 may be positioned, and/or repositioned during the surgical procedure, on the operating table (not shown) based on the surgical procedure performed.

### Systems and Methods for Guiding the Placement of a Second Camera

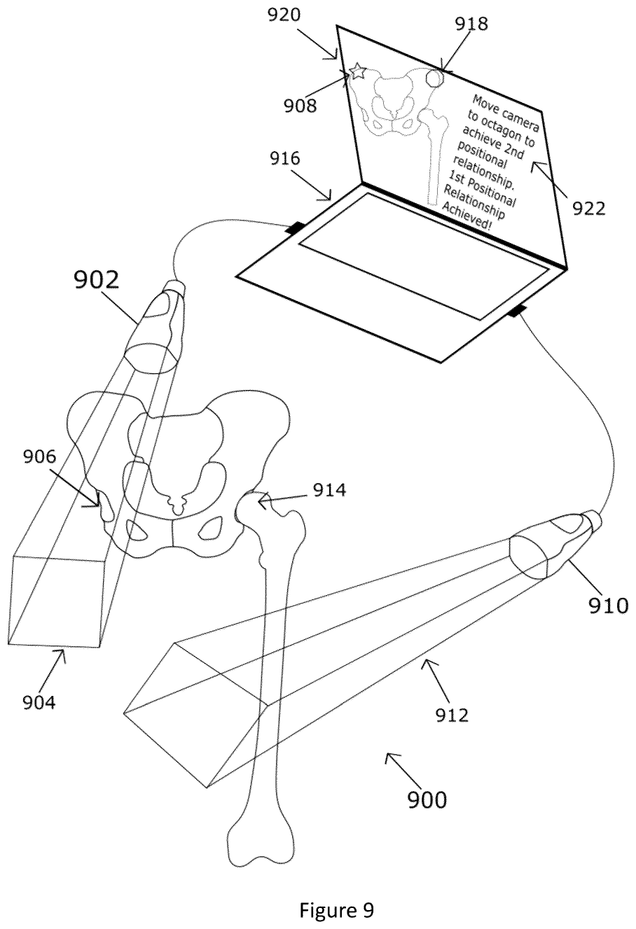

In an embodiment, a system to perform a navigated surgical procedure on a patient may comprise two or more cameras and a computing unit configured to guide the placement of one or more cameras. FIG. 9 displays the exemplary system 900 comprising a first camera 902 (similar to cameras 202 or 602) with a first field of view 904 for aligning relative to a first surgical location 906 (similar to 606) and in a first positional relationship 908 with the patient anatomy, a second camera 910 with a second field of view 912 for aligning relative to a second surgical location 914, and a computing unit 916 (similar to 214 or 616, configure as is applicable such as via software programming) configured to guide the user to position the second camera 910 (similar to 610) in a target second positional relationship 918 through a user interface 920. In this example, the first camera 902 is aligned with the first surgical location 906, the second camera 910 is not aligned with the second surgical location 914, and the user interface guides the user through a message 922 to align the second camera 910 with the second surgical location 914. The scope of this description should not be limited to two cameras aligning to two surgical locations, but two or more cameras aligning to at least one surgical location. The first camera 902 and the second camera 910 may be aligned with the same surgical location to increase the line of sight to that surgical location.

The first camera 902 and the second camera 910 may be configured to generate optical information, for example, image data, of trackers (not shown) within the respective fields of view 906 or 912. The computing unit 916 may be configured to receive the optical information from the cameras 902, 910 and process the optical information to guide the user to position the second camera 910 to the target second positional relationship through the user interface 920.

In an embodiment, the computing unit 916 may present the user interface 920 to guide the user based on the field of view 904 of the first camera 902, providing a target location to place the second camera 910 based on the amount of overlapping field of view desired by the surgical workflow and/or the user. The guiding may be based on the field of view 904 of the first camera 902, providing the user a target location to place the second camera 910 so that the second field of view 912 does not overlap with the first field of view 904.

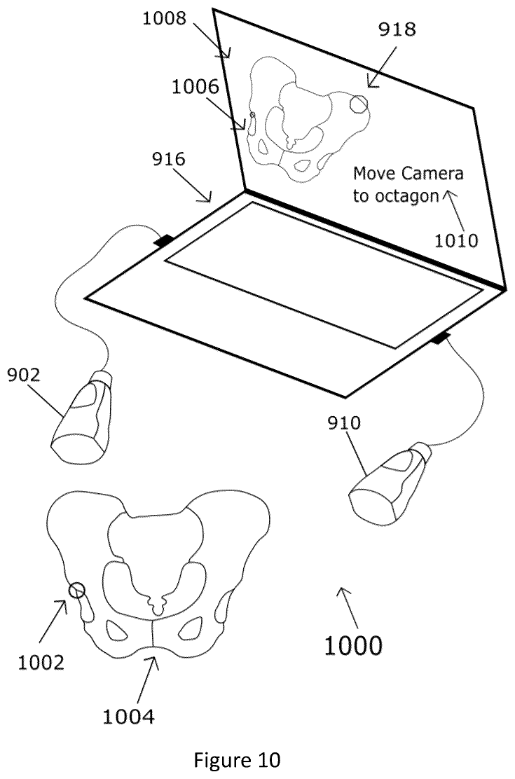

In an embodiment, the computing unit 916 may be configured to perform an image registration, defining a first coordinate system using medical images and relating those images to a plurality of landmarks on the patient anatomy, and that may be identified using the optically trackable targets. The computing unit may be configured to receive a set of patient medical images and/or segmented images (2D or 3D), perform an image registration, and determine the target second positional relationship partially based on a spatial profile of the patient anatomy. The spatial profile of the patient anatomy may include bony anatomy and/or soft tissue. FIG. 10 displays an exemplary image registration system 1000, in accordance with an embodiment, where the first camera 902 provides a first coordinate system 1002 relative to the patient anatomy 1004, the model of the patient anatomy 1006 is provided to the computing unit 916, and the user interface 1008 provides the target second positional relationship 918 for the user to position the second camera 910. The target second positional relationship 918 is based on the image registration performed with the first camera 902 and the 3D model of the patient anatomy. In the provided example, the user is informed to move the second camera 910 via a message 1010 displayed on the user interface, but the guiding may be any form of communication (e.g., visualize, audio, etc.).

The target second positional relationship 918 may be an exact positional relationship that is defined by the target registered coordinate system, based on the expected placement of the second camera 910. The target second positional relationship 918 may be a range of acceptable positional relationships. In FIG. 10, the target second positional relationship 918 may not be just a single position, but may be a multitude of positions. Within the range of acceptable positional relationships, the registered coordinate system may be a range of acceptable registered coordinate systems, and the final registered coordinate system may be calculated, for example, using a tracker (not shown). The registered coordinate system may be undefined after positioning the second camera 910 within the target second positional relationship 918, and calculated, for example, using a tracker. The target second positional relationship 918 may be a position and/or orientation of the second camera 910, or it may be a range of positions and/or orientations of the second camera 910.

The target second positional relationship 918 may be defined based on a surgical plan. The surgical plan may be defined prior to the navigated surgical procedure by a surgeon and may comprise a surgical target for the surgeon to achieve in the navigated surgical procedure. As one example, in Total Hip Arthroplasty (THA), the surgical target may comprise one or more numeric values, such as cup angle, inclination, anteversion, offset, etc., which may be useful during the placement of an acetabular cup prothesis. The surgical plan may provide the system with the optimal position to place the second camera, thus creating the target second positional relationship, based on targets the surgeon may want to achieve and/or surgical locations provided by the surgical plan.

The surgical plan may comprise a surgeon receiving a digital model (2D or 3D) identifying at least a portion of patient anatomy (e.g. via computing device (not shown). The surgeon may identify a plurality of landmarks using the computing device on the digital model of the patient anatomy and may use these landmarks as a target location(s) to identify actual locations on the patient anatomy during surgery, using, for example, a tracker, and mapping this plurality of landmarks to the corresponding plurality of landmarks on the digital model, to generate a registered coordinate system. As the navigated surgical procedure now directly matches the digital model through the registered coordinate system, the surgical targets identified in the surgical plan may be visualized and achieved through the navigated surgical procedure.

The computing unit 916 may receive the surgical plan through a user entering data into the computing unit and/or a surgical planning software (e.g. via communication or retrieved from a storage unit, etc.). The surgical plan may be displayed by the computing unit 916 through a user interface, displaying the surgical targets and/or surgical workflow. The user interface may display medical images from the surgical plan with surgical targets overlayed on the patient anatomy and/or target visualizations (e.g., surgical targets and/or target positional relationships).

The user interface may guide the user to place the second camera 910 in the target second positional relationship 918 based on the surgical plan. In one example, the surgical plan may define a target surgical location (e.g. 914), and the user interface may guide the user to place the second camera 910 in an area where the second field of view 912 would cover the target surgical location 914. The user interface may provide an exact target second positional relationship 918 or an acceptable range of target second positional relationships. The user interface may provide feedback (e.g., sound and/or visualization) to inform the user when the target second positional relationship 918 has been achieved. In another example, the surgical plan may include the planned first positional relationship and the target second positional relationship 918 and update the target second positional relationship 918 based on the image registration with the first camera 902 and/or update the planned first positional relationship based on the actual first positional relationship defined by the image registration.

The first camera 902 and the second camera 910 may generate a first set of optical information, for example, image data, of a tracker within the overlapping field of view, and the user interface may inform the user where to place the first camera 902 to generate the first positional relationship and/or the second camera 910 to generate the second positional relationship, based on the pose of the tracker from the first set of optical information. The tracker may comprise two or more subsets of optically trackable targets, which may be advantageous for a non-overlapping field of view and may generate the first set of image data in each of the first camera 902 and the second camera 910. The user interface may guide the user to place the first camera 902 and/or the second camera 910 based on the pose of the tracker from the first set of image data to optimize the amount of overlapping field of view and/or optimize the amount of non-overlapping field of view. The user interface may inform the user where to position the first 902 and/or second camera 910 based on the pose of the tracker, ensuring that each of the first and second fields of view contains the tracker. The tracker may be positioned over the target surgical location, using bone fixation hardware or a rigid body fixed to the patient, which when contained within the first and/or second cameras field of view, provides the target first and/or second positional relationship of the first 902 and/or second camera 910.

In an embodiment, the first camera 902 comprises a first inertial sensor (not shown) and the second camera comprises 910 a second inertial sensor (not shown), and the guiding may be based on measurements from the respective inertial sensors. In one example, the measurements from the first inertial sensor and the second inertial sensor may provide gravity vectors, pointing in the direction of gravity. The user interface may guide the user to place the second camera 910 at a target orientation, and that target orientation may be the same or approximate orientation as the first camera, or vice versa. The target orientation may be based on a surgical plan, for example, if the first camera 902 placed at the target orientation, and thus a first positional relationship, would optimize the line of sight to the first surgical location, the user interface would guide the user to place the first camera 902 at that target orientation. The target orientation may be based on a surgical plan and may guide the user to position the camera at a position or orientation that may be used to inform the system of a surgical workflow and/or location. The guiding may be based on a target relative orientation between the first camera 902 and the second camera 910. The targets provided from the measurements from the first and/or second inertial sensor may be an exact positional relationship or a range of acceptable positional relationships.

The first positional relationship may be a fixed positional relationship in which the first camera 902 is rigidly fixed to the patient or a non-fixed positional relationship where the system (e.g. 600, 900 or 1000) comprises a patient reference tracker. The patient reference tracker may be configured to have a fixed rigid positional relationship with the patient via bone fixation hardware (3.g. 404) and be within the first field of view of the first camera (e.g. 602 or 902, etc). The fixed rigid relationship defines a coordinate system relative to the patient reference tracker, instead of relative to either the first camera 902 or the second camera 910. The bone fixation hardware (e.g. 404) may comprise a fixed attachment to the patient reference tracker or a repeatable attachment to the patient reference tracker. The user interface may guide the user where to place the first camera based on the patient reference tracker and the guide may be based on a planned target positional relationship.

The user interface may guide the user to place the first camera and/or the second camera in a first positional relationship and/or second positional relationship based on the patient reference tracker. In one example, the first positional relationship may be established, and a first coordinate system may be defined relative to the patient reference tracker. The user interface may guide the user where to create the second positional relationship of the second camera to minimize the amount of overlapping field of view so that the overall field of view of the two or more cameras is optimized but there is enough of an overlapping field of view to contain the patient reference tracker in both the first second cameras field of view, which may be used to generate the registered coordinate system and/or update the registered coordinate system. In another example, the surgical plan may consist of a range of acceptable positional relationships for the first camera and/or the second camera, relative to the patient reference tracker, and the user interface may guide the user to position the first camera and/or second camera in these regions. These regions may be based on the patient reference tracker, surgical plan, etc.

In addition to system, apparatus and method aspects, there is provided a tangible non-transitory computer readable medium storing instructions which when executed by a computing device configure the computing device to perform any of the methods as described.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

Throughout the description and claims of this specification, the word “comprise” and “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise. By way of example and without limitation, references to a computing device comprising a processor and/or a storage device includes a computing device having multiple processors and/or multiple storage devices. Herein, “A and/or B” means A or B or both A and B.

Features, integers characteristics, compounds, chemical moieties or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

Various features and aspects will be understood from the following numbered Statements.

Statement 1: A system for registering two or more cameras for use in a navigated surgical procedure comprising:
a. a first camera providing a first field of view, and configured for being positioned in a first positional relationship with a patient;
b. a second camera providing a second field of view, and configured for being positioned independently of the first camera in a second positional relationship with the patient;
c. a tracker for simultaneous viewing by the first and second cameras; and
d. a computer storage device storing instructions, which when executed by a processor of a computing device, cause the computing device to:
   i. receive synchronized images of the tracker within the overlapping field of view from the first and second cameras;
   ii. measure the pose of the tracker relative to the respective camera coordinate systems to calculate a registered coordinate system relative to both cameras; and
   iii. provide surgical navigation relative to the registered coordinate system with the tracker in both the overlapping and non-overlapping fields of view.

Statement 2: The system of Statement 1, wherein the first and second fields of view partially overlap, and the tracker is simultaneously viewed when in the overlapping field of view.

Statement 3: The system of Statement 1 or 2, wherein the tracker comprises two or more subsets of features for measuring the pose, the two or more subsets having a fixed rigid relationship, and the computer storage device providing the fixed rigid relationship in memory.

Statement 4: The system of any one of Statements 1 to 3, wherein the tracker is configured to be simultaneously viewed by both cameras wherein each camera views a respective subset of features, the camera fields of view not necessarily overlapping.

Statement 5: The system of any one of Statements 1 to 5, wherein the first and second cameras comprise respective first and second inertial sensors, and wherein the calculating the registered coordinate system comprises measuring a direction of gravity with respect to each respective camera and using the measured directions of gravity to calculate the registered coordinate system.

Statement 6: The system of Statement 5, wherein the direction of gravity from respective cameras is used to calculate 2 degrees of freedom of the registered coordinate system, and the remaining degrees of freedom are calculated using the synchronized images.

Statement 7: The system of any one of Statements 1 to 6, wherein the first and second cameras comprise respective first and second inertial sensors, and wherein during provision of surgical navigation, the computing device is configured to receive data from the inertial sensors and measure relative movement of the cameras by comparing the differences between the inertial sensor measurements.

Statement 8: The system of Statement 7, wherein, upon detection of relative movement of the cameras, the computing device is configured to invalidate the registered coordinate system, notify a user via a user interface that the registered coordinate system is invalidated, and provide a user interface to guide the user to perform reregistration.

Statement 9: The system of any one of Statements 1 to 8, wherein the first positional relationship and/or the second positional relationship is a fixed relationship in which the respective camera is rigidly fixed to the patient

Statement 10: The system of any one of Statements 1 to 9, wherein the first positional relationship and/or the second positional relationship is not fixed, and wherein the system comprises a patient reference tracker having a fixed rigid relationship with the patient, and the patient reference tracker configured to be attached to the patient and within the field of view of the respective first and/or second camera.

Statement 11: The system of Statements 9 or 10, wherein one camera has a fixed positional relationship and the other camera has a positional relationship determined by the pose of a patient reference tracker.

Statement 12: The system of any one of Statements 9 to 11, wherein upon detection of movement of the patient reference tracker, the computing unit is configured to invalidate the registered coordinate system, notify a user via a user interface that the registered coordinate system is invalidated, and provide a user interface to guide the user to perform reregistration.

Statement 13: The system of any one of Statements 1 to 12, wherein the computing unit is further configured to provide a user interface guiding a user to move the tracker into a location for simultaneous viewing by the first and second cameras.

Statement 14: The system of any one of Statements 1 to 13, wherein the computing unit is configured to display via a user interface a merged image feed of both cameras based on the registered coordinate system.

Statement 15: The system of statement 14, wherein the merged image feed is based on the perspective of the first camera or the second camera, or another perspective relative to the registered coordinate system.

Statement 16: The system of any one of Statements 1 to 15, wherein the computing unit is configured to perform an anatomical registration and/or image registration in which locations of a plurality of landmarks are received based on image data of a registration tracker, the image data received from the first camera for a subset of the plurality and from the second camera for another subset of the plurality, and wherein the registration tracker is not within the overlapping field of view.

Statement 17: The system of any one of Statements 1 to 16, wherein during surgical navigation, as a tracker transitions from the first field of view into the second field of view (or vice versa), the calculation of the pose of the tracker within the second field of view is based on the pose of the tracker in the first field of view (or vice versa) and the registered coordinate system.

Statement 18: A system for performing a navigated surgery at two or more locations on a patient, the system comprising: (a) a first camera with a first field of view for aligning relative to the first surgical location, and configured to generate image data of trackers; (b) a second camera with a second field of view for aligning relative to the second surgical location and configured to generate image data of trackers; and (c) a computer storage device storing instructions, which when executed by a processor of a computing device, cause the computing device to: (i) receive image data from the first and second camera; (ii) detect trackers within the respective fields of view of the first and second cameras; (iii) provide a surgical workflow for the navigated surgery via a user interface; and (iv) modify the surgical workflow responsive to the detected trackers.

Statement 19: The system of Statement 18, wherein the trackers comprise multiple uniquely identifiable trackers.

Statement 20: The system of Statement 18 or 19, wherein detecting trackers comprises detecting if a tracker is within the field of view, and if so, identifying the tracker(s) and/or measuring the pose of the tracker(s).

Statement 21: The system of any one of Statements 18 to 20, wherein the navigated surgery is an image-based navigation, and wherein the modifying of the surgical workflow comprises changing the patient images that are displayed on the user interface.

Statement 22: A system for performing a navigated surgical procedure on a patient comprising: (a) a first camera with a first field of view for aligning relative to a first surgical location, and configured to generate image data of trackers, and in a first positional relationship with the patient; (b) a second camera with a second field of view for aligning relative to the second surgical location and configured to generate image data of trackers; and (c) a computer storage device storing instructions, which when executed by a processor of a computing device, configure the computing device to provide a user interface to guide the user to position the second camera in a target second positional relationship.

Statement 23: The system of claim 1, wherein the computer is further configured to perform an image registration and the guiding is based on the image registration.

Statement 24: The system of Statement 23, wherein the image registration comprises receiving a patient image and/or segmented image (2D or 3D) and the target second positional relationship is determined partially based on the spatial profile of the patient's anatomy (including bony anatomy and/or soft tissues).

Statement 25: The system of any one of Statements 22 to 24, wherein the target second positional relationship is a range of acceptable positional relationships.

Statement 26: The system of any one of Statements 22 to 25, wherein the target second positional relationship is based on a surgical plan.

Statement 27: The system of any one of Statements 22 to 26, wherein the first camera and second camera generate first images of a tracker, and the guiding is based on the pose of the tracker as determined by the first images.

Statement 28: The system of any one of Statements 22 to 27, wherein the first and second cameras comprise respective first and second inertial sensors, and wherein the guiding is based on measurements from the respective inertial sensors.

Statement 29: The system of any one of Statements 22 to 28, wherein the first positional relationship is one of: a fixed relationship in which the respective camera is rigidly fixed to the patient; and a non-fixed positional relationship wherein the system comprises a patient reference tracker having a fixed rigid relationship with the patient, and the patient reference tracker configured to be attached to the patient and within the field of view of the first camera.

It will be understood that corresponding computer implemented method aspects and/or computer program product aspects are also disclosed. A computer program product, for example, comprises a storage device storing computer readable instructions that when executed by at least one processor of a computing device causes the computing device to perform operations of a computer implemented method.

What is claimed is:

1. A system for performing a navigated surgical procedure on a patient comprising:
a. a first camera with a first field of view for aligning relative to a first surgical location, and configured to generate image data of trackers, and in a first positional relationship with the patient;
b. a second camera with a second field of view for aligning relative to the second surgical location and configured to generate image data of trackers; and
c. a computer storage device storing instructions, which when executed by a processor of a computing device, configure the computing device to:
i. provide a user interface to guide the user to position the second camera in a target second positional relationship.

2. The system of claim 1, wherein the computer is further configured to perform an image registration and the guiding is based on the image registration.

3. The system of claim 2, wherein the image registration comprises receiving a patient image and/or segmented image, and the target second positional relationship is determined partially based on the spatial profile of the patient's anatomy.

4. The system of claim 1, wherein the target second positional relationship is a range of acceptable positional relationships.

5. The system of claim 1, wherein the target second positional relationship is based on a surgical plan.

6. The system of claim 1, wherein the first camera and second camera generate first images of a tracker, and the guiding is based on the pose of the tracker as determined by the first images.

7. The system of claim 1 wherein the first and second cameras comprise respective first and second inertial sensors, and wherein the guiding is based on measurements from the respective inertial sensors.

8. The system of claim 1, wherein the first positional relationship is one of: a fixed relationship in which the respective camera is rigidly fixed to the patient; and a non-fixed positional relationship wherein the system comprises a patient reference tracker having a fixed rigid relationship with the patient, and the patient reference tracker configured to be attached to the patient and within the field of view of the first camera.

9. A computer implemented method for performing a navigated surgical procedure on a patient, wherein a first camera is in a first positional relationship with the patient, the first camera having a first field of view aligned relative to a first surgical location, and the first camera configured to generate image data of trackers, the method comprising:
a. defining and presenting a user interface to guide a user to position a second camera in a target second positional relationship with the patient, the second camera having a second field of view for aligning relative to a second surgical location, and the second camera configured to generate image data of trackers; and
b. receiving respective image data from each of the first camera and the second camera to perform the navigated surgical procedure.

10. The method of claim 9, comprising performing an image registration, and wherein defining the user interface to guide is based on the image registration.

11. The method of claim 10, wherein the image registration comprises receiving a patient image and/or segmented image in two dimensions or three dimensions, and the method comprises determining the target second positional relationship partially based on a spatial profile of an anatomy of the patient.

12. The method of claim 9, wherein the target second positional relationship is a range of acceptable positional relationships.

13. The method of claim 9, wherein the target second positional relationship is based on a surgical plan.

14. The method of claim 9, wherein the first camera and second camera generate first images of a tracker, and wherein defining the user interface to guide is based on the pose of the tracker as determined by the first images.

15. The method of claim 9, wherein the first and second cameras comprise respective first and second inertial sensors, and wherein the method comprises receiving measurements from each of the first and second cameras and wherein defining the user interface to guide is based on the measurements.

16. The method of claim 9, wherein the first positional relationship is one of: a fixed relationship in which the first camera is rigidly fixed to the patient; and a non-fixed positional relationship in which a patient reference tracker has a fixed rigid relationship with the patient, and the patient reference tracker is configured to attach to the patient and within the field of view of the first camera.
